(19) United States
(12) Patent Application Publication
(10) Pub. No.: US 2002/0165141 A1
(43) Pub. Date: Nov. 7, 2002

(54) Methods for promoting dendritic cell proliferation or differentiation

(76) Inventors: Gere S. diZerega, Pasedena, CA (US); Kathleen E. Rodgers, Long Beach, CA (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 09/900,936

(22) Filed: Jul. 9, 2001

Related U.S. Application Data
(60) Provisional application No. 60/217,951, filed on Jul. 13, 2000.

(51) Int. Cl. A61K 38/16

(52) U.S. Cl. 514/12

(57) ABSTRACT

The present invention provides methods, pharmaceutical compositions, and kits for promoting dendritic cell proliferation and differentiation, through the use one or more of renin, angiotensinogen, angiotensinogen converting enzyme (ACE), neutral endopeptidase, angiotensin I (AI), AI analogues, AI fragments and analogues thereof, angiotensin II (AII), AII analogues, AII fragments or analogues thereof or AII AT₂ type 2 receptor agonists, angiotensin converting enzyme inhibitors (ACE inhibitors). Such methods are useful, for example, for promoting tumor vaccine production, or for improving the efficacy of a tumor vaccine.

### CROSS REFERENCE

This application claims priority to U.S. patent application Ser. No. 60/217,951 filed Jul. 13, 2001.

### FIELD OF THE INVENTION

This application relates to the fields of immunology and cell biology.

### BACKGROUND OF THE INVENTION

Modulation of the immune system has emerged as an attractive treatment option for cancer patients. Unlike traditional cancer treatments such as radiation therapy or chemotherapy, the immune system can discriminate between healthy and cancer cells. Once activated, the cells of the immune system are highly efficient at killing target cells.

The anticancer effects of the immune system are primarily accomplished by the cell-mediated component of the immune system, and most of these effects are regulated by T cells. (Chen and Wu, J. Biomed. Sci. 5:231-252 (1998). Activated T cells may function directly as effector cells, by the lysis of tumor cells, or through the release of cytokines capable of interfering with the propagation of tumors. Also, the function of “non-specific” effector cells such as macrophages, natural killer cells and granulocytes are critically regulated by T-cell-derived factors. T cells also possess the ability to recognize tumor-associated antigens (TAAs). TAAs serve as targets by which T cells can distinguish cancerous from non-cancerous tissues. (Chen and Wu (1998); Gouttefangeas and Rammensee, Nature Biotech. 18:491-492 (2000)). Since most cancer patients do not mount efficient T-cell responses against their tumors, immunotherapies preferably induce cancer-destroying T cells in patients.

T cells must first be activated before they can mediate anticancer effects. In activation, an antigen-presenting cell (APC) “presents” the TAA to a T cell, through cell-cell contact. APCs have at least two distinct pathways for the processing of antigens recognized by T cells. CD8+ cytotoxic T lymphocytes (CTLs) recognize antigens that are presented on major histocompatibility complex class I (MHC-I) molecules. (Chen and Wu (1998); Gouttefangeas and Rammensee (2000)) MHC-I molecules are expressed on most cells of the body. In contrast, CD4+T helper cells identify peptide antigens that are presented on MHC class II molecules (MHC-II). MHC-II molecules are predominantly expressed on specialized APCs, such as macrophages, dendritic cells, and activated B cells. TAAs, when efficiently presented by APCs to both CD8+CTL and CD4+helper T cells, are capable of inducing potent T-cell-mediated immunity. Therefore, the ideal tumor vaccine would enhance both CD8+CTL and CD4+helper T cell responses by delivering a TAA into both the MHC-I and MHC-II pathways of antigen presentation. (Chen and Wu (1998); Gouttefangeas and Rammensee (2000))

Many different types of tumor vaccines have been developed. (For example, see Chen and Wu (1998)). Vector-based vaccines employ either viral vectors, such as adenovirus or vaccinia virus, or bacteria, such as Listeria or Salmonella, to deliver the TAA or a cytokine to the APC. (See, for example, U.S. Pat. No. 6,051,428.) Peptide-based vaccines typically comprise a synthetic peptide that corresponds to an epitope recognized by a CTL. Protein-based vaccines involve the use of entire proteins, and are useful when the CTL epitopes of a TAA are undefined. DNA vaccines involve the administration of naked DNA that encodes a TAA. Other potential tumor vaccines include tumor-specific antibodies, or antibodies to negative regulators of T-cell activation. (See, for example, U.S. Pat. No. 5,798,100.)

An especially important class of tumor vaccines are cell-based vaccines. These can be divided into two broad categories: dendritic cell-based vaccines and tumor cell-based vaccines. (Chen and Wu (1998)) Dendritic cells are extremely potent APCs that are specialized to prime helper and killer T cells in vivo. Therefore, vaccine strategies employing dendritic cells generated ex vivo to enhance T-cell-mediated immunity against tumors have become a desirable option. In this approach, dendritic cells are first generated from hematopoietic progenitor cells in the presence of various cytokines, mainly GM-CSF and Flt3-L. They are then either pulsed with TAA peptides or proteins, transduced with genes coding for TAAs, pulsed with tumor extracts or tumor-derived RNAs, or fused with tumor cells, before replacement into the patient. (Chen and Wu (1998); Lotze et al., Cancer J. Sci. Am. 2000; 6 (suppl. 1):S61-S66).

Tumor-cell based vaccines, in contrast, do not involve ex vivo methods, but rather aim to increase the numbers and function of dendritic cells in vivo. The general strategy of tumor-cell based vaccines is to deliver in vivo a tumor cell engineered to express a cytokine that stimulates the differentiation of dendritic cells, e.g., GM-CSF or Flt3-L. The goal is to create very high concentrations of the cytokine local to the injected tumor cells. Such vaccines include autologous GM-CSF transduced cell-based vaccines, allogeneic GM-CSF transduced cell-based vaccines, or bystander GM-CSF releasing microspheres or cells. (Chen and Wu (1998)).

Despite the large number of cancer immunotherapy approaches that have been developed, effective immunotherapy has remained elusive. (Gouttefangeas and Rammensee (2000)) Thus, there is a need in the art for improved cancer immunotherapy methods.

### SUMMARY OF THE INVENTION

The present invention provides methods, pharmaceutical compositions, and kits for promoting dendritic cell proliferation and differentiation, comprising using or administering an amount effective to promote dendritic cell proliferation or differentiation of renin, angiotensinogen, angiotensinogen converting enzyme (ACE), neutral endopeptidase, angiotensin I (AI), AI analogues, AI fragments and analogues thereof, angiotensin II (AII), AII analogues, AII fragments or analogues thereof or AII AT₂ type 2 receptor agonists, angiotensin converting enzyme inhibitors (ACE inhibitors), either alone, combined, or in further combination with other compounds for promoting dendritic cell proliferation or differentiation. Such methods are useful, for example, by promoting tumor vaccine production or for improving the efficacy of a tumor vaccine.

### BRIEF DESCRIPTION OF THE FIGURES

FIG. 1 is a bar graph summarizing the effect of AII on dendritic cell proliferation.

FIG. 2 is a bar graph summarizing the effect of AII (1-7) on dendritic cell proliferation.

FIG. 3 is a bar graph summarizing the effect of AII(1-5) on dendritic cell proliferation.

FIG. 4 is a bar graph summarizing the effect of IL-6 and AII on dendritic cell proliferation.

FIG. 5 is a bar graph summarizing the effect of IL-6 and AII(1-7) on dendritic cell proliferation.

FIG. 6 is a bar graph summarizing the effect of IL-6 and AII(1-5) on dendritic cell proliferation.

FIG. 7 is a bar graph summarizing the effect of GM-CSF and AII on dendritic cell proliferation.

FIG. 8 is a bar graph summarizing the effect of GM-CSF and AII(1-7) on dendritic cell proliferation.

FIG. 9 is a bar graph summarizing the effect of GM-CSF and AII(1-5) on dendritic cell proliferation.

FIG. 10 is a bar graph summarizing the effect of IL-6, GM-CSF, Flt3 ligand, and AII on dendritic cell proliferation.

FIG. 11 is a bar graph summarizing the effect of IL-6, GM-CSF , Flt3 ligand, and AII(1-7) on dendritic cell proliferation.

FIG. 12 is a bar graph summarizing the effect of IL-6, GM-CSF, Flt3 ligand, and AII(1-5) on dendritic cell proliferation.

FIG. 13 is a bar graph summarizing the effect of angiotensin peptides on antigen presentation by dendritic cells to BZ3 T cell hybridomas.

FIG. 14 is a bar graph summarizing the effect of angiotensin peptides and IL-6 on antigen presentation by dendritic cells to BZ3 T cell hybridomas.

### DETAILED DESCRIPTION OF THE PREFERRED EMBODIMENTS All cited patents, patent applications and references are hereby incorporated by reference in their entirety.

Unless otherwise indicated, the term “angiotensin converting enzyme inhibitors” or “ACE inhibitors” includes any compound that inhibits the conversion of the decapeptide angiotensin I to angiotensin II, and include but are not limited to alacepril, alatriopril, altiopril calcium, ancovenin, benazepril, benazepril hydrochloride, benazeprilat, benzazepril, benzoylcaptopril, captopril, captopril-cysteine, captopril-glutathione, ceranapril, ceranopril, ceronapril, cilazapril, cilazaprilat, converstatin, delapril, delapril-diacid, enalapril, enalaprilat, enalkiren, enapril, epicaptopril, foroxymithine, fosfenopril, fosenopril, fosenopril sodium, fosinopril, fosinopril sodium, fosinoprilat, fosinoprilic acid, glycopril, hemorphin-4, idapril, imidapril, indolapril, indolaprilat, libenzapril, lisinopril, lyciumin A, lyciumin B, mixanpril, moexipril, moexiprilat, moveltipril, muracein A, muracein B, muracein C, pentopril, perindopril, perindoprilat, pivalopril, pivopril, quinapril, quinapril hydrochloride, quinaprilat, ramipril, ramiprilat, spirapril, spirapril hydrochloride, spiraprilat, spiropril, spiropril hydrochloride, temocapril, temocapril hydrochloride, teprotide, trandolapril, trandolaprilat, utibapril, zabicipril, zabiciprilat, zofenopril and zofenoprilat. (See for example Jackson, et al., Renin and Angiotensin in Goodman & Gilman's The Pharmacological Basis of Therapeutics, 9th ed., eds. Hardman, et al. (McGraw Hill, 1996); and U.S. Pat. No. 5,977,159.)

Unless otherwise indicated, the term “active agents” as used herein refers to the group of compounds comprising renin (converts angiotensinogen to AI), angiotensinogen, angiotensinogen converting enzyme (ACE) (converts AI to AII), neutral endopeptidase (converts AI to AII(1-7)), angiotensin I (AI), AI analogues, AII fragments and analogues thereof, angiotensin II (AII), AII analogues, AII fragments or analogues thereof or AII AT₂ type 2 receptor agonists, either alone, combined, or in further combination with other compounds, for promoting tumor vaccine production or improving the efficacy of a tumor vaccine, such as cytokines, cytokine-encoding nucleic acid molecules, polycations (such as polylysine), heat shock proteins, viral-like particles, and incomplete Freund's adjuvant (IFA).

As used herein, “proliferation” encompasses both proliferation and proliferation with accompanying differentiation.

As used herein, “differentiation” encompasses includes both entry into a specific lineage pathway and functional activation of differentiated cells, including increased ability of dendritic cells to present antigen and thus potentiate immune responses.

As used herein, “dendritic cells” refer to bone marrow-derived antigen-presenting cells, and specifically include Langerhans cells. Such dendritic cells can be found throughout the body, including but not limited to blood, spleen, lymphatic system, lymphoid organs, skin, and sub-mucosally.

As used herein, the phrase “promoting tumor vaccine production” includes, but is not limited to facilitating the production of tumor vaccines, increasing the amount of tumor vaccine produced, increasing the speed of tumor vaccine production, improving the efficiency of action of the tumor vaccine, increasing the speed of eliciting of an immune response following administration of a tumor vaccine, and increasing the duration of an immune response following administration of a tumor vaccine. Such promotion or improvement can be via any means by which tumor vaccines are produced, including but not limited to vector-based vaccines, peptide-based vaccines, protein-based vaccines, DNA vaccines, dendritic cell-based vaccines, tumor cell vaccines, and vaccine delivery via microspheres. The phrase further encompasses any mechanism by which such promotion is achieved, including but not limited to enhancement of CD8+CTL response; enhancement of CD4+ helper T cell response; tumor cell lysis; stimulation of cytokine release; modulation of the proliferation or activity of macrophages, natural killer cells, granulocytes, dendritic cells, and antigen presenting cells, including but not limited to B cells.

As used herein, the term “cancer” and “tumor” include all forms of cancer and tumors.

As used herein, the term “cytokine” includes, but is not limited to hematopoietic growth factors including but not limited to granulocyte macrophage colony stimulating factor (GM-CSF) and macrophage colony stimulating factor (M-CSF); fms-like tyrosine kinase 3 Ligand (Flt3-L), tumor necrosis factor family molecules including but not limited to tumor necrosis factor-α (TNF-α); interleukins including but not limited to interleukin-1 (IL-1), IL-2, IL-4, IL-6, IL-12, interferons, immunoglobulin superfamily molecules, chemokines, and active fragments thereof.

U.S. Pat. No. 5,015,629 to DiZerega (the entire disclosure of which is hereby incorporated by reference) describes a method for increasing the rate of healing of wound tissue, comprising the application to such tissue of angiotensin II (AII) in an amount which is sufficient for said increase. The application of AII to wound tissue significantly increases the rate of wound healing, leading to a more rapid re-epithelialization and tissue repair. The term AII refers to an octapeptide present in humans and other species having the sequence Asp-Arg-Val-Tyr-Ile-His-Pro-Phe [SEQ ID NO:1]. The biological formation of angiotensin is initiated by the action of renin on the plasma substrate angiotensinogen (Circulation Research 60:786-790 (1987); Clouston et al., Genomics 2:240-248 (1988); Kageyama et al., Biochemistry 23:3603-3609; Ohkubo et al., Proc. Natl. Acad. Sci. 80:2196-2200 (1983)); all references hereby incorporated in their entirety). The substance so formed is a decapeptide called angiotensin I (AI) which is converted to AII by the converting enzyme angiotensinase which removes the C-terminal His-Leu residues from AI, Asp-Arg-Val-Tyr-Ile-His-Pro-Phe-His-Leu [SEQ ID NO:37]. AII is a known pressor agent and is commercially available.

Studies have shown that AII increases mitogenesis and chemotaxis in cultured cells that are involved in wound repair, and also increases their release of growth factors and extracellular matrices (dizerega, U.S. Pat. No. 5,015,629; Dzau et. al., J Mol. Cell. Cardiol. 21:S7 (Supp III) 1989; Berk et. al., Hypertension 13:305-14 (1989); Kawahara, et al., BBRC 150:52-9 (1988); Naftilan, et al., J Clin. Invest. 83:1419-23 (1989); Taubman et al., J Biol. Chem. 264:526-530 (1989); Nakahara, et al., BBRC 184:811-8 (1992); Stouffer and Owens, Circ. Res. 70:820 (1992); Wolf, et al., Am. J Pathol. 140:95-107 (1992); Bell and Madri, Am. J. Pathol. 137:7-12 (1990)). In addition, AII was shown to be angiogenic in rabbit corneal eye and chick chorioallantoic membrane models (Fernandez, et al., J Lab. Clin. Med. 105:141 (1985); LeNoble, et al., Eur. J. Pharmacol. 195:305-6 (1991)).

We have previously demonstrated that angiotensinogen, angiotensin I (AI), AI analogues, AI fragments and analogues thereof, angiotensin II (AII), AII analogues, AII fragments or analogues thereof; AII AT₂ type 2 receptor agonists are effective in accelerating wound healing and the proliferation of certain cell types. See, for example, co-pending U.S. patent application Ser. Nos. 09/012,400; 09/264,563; 09/287,674; 09/307,940; 09/307,940; 09/255,136; 09/250,703; 09/246,525; 09/266,293; 09/332,582; and 09/352,191, as well as U.S. Pat. Nos. 5,015,629; 5,629,292; 5,716,935; 5,834,432; 5,955,430; 6,177,407; 6,239,109; and 6,248,587; all references herein incorporated in their entirety.

The effect of AII on a given cell type has been hypothesized to be dependent, in part, upon the AII receptor subtypes the cell expresses (Shanugam et al., Am. J Physiol. 268:F922-F930 (1995); Helin et al., Annals of Medicine 29:23-29 (1997); Bedecs et al., Biochem J. 325:449-454 (1997)). These studies have shown that AII receptor subtype expression is a dynamic process that changes during development, at least in some cell types. AII activity is typically modulated by either or both the AT1 and AT2 AII receptors. However, AII has recently been shown to stimulate proliferation of primary human keratinocytes via a non-AT1, non-AT2 receptor. (Steckelings et al., Biochem. Biophys. Res. Commun. 229:329-333 (1996)). These results underscore the cell-type (ie: based on receptor expression) specific nature of AII activity.

Many studies have focused upon AII(1-7) (AII residues 1-7) or other fragments of AII to evaluate their activity. AII(1-7) elicits some, but not the full range of effects elicited by AII. (Pfeilschifter, et al., Eur. J Pharmacol. 225:57-62 (1992); Jaiswal, et al., Hypertension 19(Supp. II):II-49-II-55 (1992); Edwards and Stack, J Pharmacol. Exper. Ther. 266:506-510 (1993); Jaiswal, et al., J. Pharmacol. Exper. Ther. 265:664-673 (1991); Jaiswal, et al., Hypertension 17:1115-1120 (1991); Portsi, et a., Br. J Pharmacol. 111:652-654 (1994)).

Other data suggests that the AII fragment AII(1-7) acts through a receptor(s) that is distinct from the AT1 and AT2 receptors which modulate AII activity. (Ferrario et al., J. Am. Soc. Nephrol. 9:1716-1722 (1998); Iyer et al., Hypertension 31:699-705 (1998); Freeman et al., Hypertension 28:104 (1996); Ambuhl et al., Brain Res. Bull. 35:289 (1994)). Thus, AII(1-7) activity on a particular cell type cannot be predicted based solely on the effect of AII on the same cell type. In fact, there is some evidence that AII(1-7) often opposes the actions of AII. (See, for example, Ferrario et al., Hypertension 30:535-541 (1997).)

Based on the above, there would be no expectation by one of skill in the art that the active agents of the invention, or the DNA or RNA encoding any of these, could be used for promoting tumor vaccine production.

A peptide agonist selective for the AT2 receptor (AII has 100 times higher affinity for AT2 than AT1) is p-aminophenylalanine6-AII [“(p-NH₂-Phe)6-AII)”], Asp-Arg-Val-Tyr-Ile-Xaa-Pro-Phe [SEQ ID NO.36] wherein Xaa is p-NH₂-Phe (Speth and Kim, BBRC 169:997-1006 (1990). This peptide gave binding characteristics comparable to AT2 antagonists in the experimental models tested (Catalioto, et al., Eur. J. Pharmacol. 256:93-97 (1994); Bryson, et al., Eur. J Pharmacol. 225:119-127 (1992).

The effects of AII and AII receptor antagonists have been examined in two experimental models of vascular injury and repair which suggest that both AII receptor subtypes (AT1 and AT2) play a role in wound healing (Janiak et al., Hypertension 20:737-45 (1992); Prescott, et al., Am. J Pathol. 139:1291-1296 (1991); Kauffinan, et al., Life Sci. 49:223-228 (1991); Viswanathan, et al., Peptides 13:783-786 (1992); Kimura, et al., BBRC 187:1083-1090 (1992)).

As hereinafter defined, a preferred class of AT2 agonists for use in accordance with the present invention comprises AII analogues or active fragments thereof having p-NH-Phe in a position corresponding to a position 6 of AII. In addition to peptide agents, various nonpeptidic agents (e.g., peptidomimetics) having the requisite AT2 agonist activity are further contemplated for use in accordance with the present invention.

The active AII analogues, fragments of AII and analogues thereof of particular interest in accordance with the present invention comprise a sequence of at least three contiguous amino acids of groups R¹-R⁸ in the sequence of general formula I

R¹-R²-R³-R⁴-R⁵-R⁶-R⁷-R⁸

wherein R¹ is selected from the group consisting of H, Asp, Glu, Asn, Acpc (1-aminocyclopentane carboxylic acid), Ala, Me²Gly, Pro, Bet, Glu(NH₂), Gly, Asp(NH₂) and Suc, or is absent,

R² is selected from the group consisting of Arg, Lys, Ala, Cit, Om, Ser(Ac), Sar, D-Arg and D-Lys,

R³ is selected from the group consisting of Val, Ala, Leu, norLeu, Ile, Gly, Lys, Pro, Aib, Acpc and Tyr;

R⁴ is selected from the group consisting of Tyr, Tyr(PO₃)₂, Thr, Ser, homoSer, azaTyr, and Ala;

R⁵ is selected from the group consisting of Ile, Ala, Leu, norLeu, Val and Gly;

R⁶ is selected from the group consisting of His, Arg or 6-NH₂-Phe;

R⁷ is selected from the group consisting of Pro or Ala; and

R⁸ is selected from the group consisting of Phe, Phe(Br), Ile and Tyr, excluding sequences including R⁴ as a terminal Tyr group.

Compounds falling within the category of AT2 agonists useful in the practice of the invention include the AII analogues set forth above subject to the restriction that R⁶ is p-NH₂-Phe.

In alternate embodiments, the active agents comprise a sequence of at least four, five, six, seven, or eight contiguous amino acids of groups R¹-R⁸ in the sequence of general formula I. In a further alternative, the active agents consist of a sequence of at least four, five, six, seven, or eight contiguous amino acids of groups R¹-R⁸ in the sequence of general formula I.

Particularly preferred combinations for R¹ and R² are Asp-Arg, Asp-Lys, Glu-Arg and Glu-Lys. Particularly preferred embodiments of this class include the following: AIII or AII(2-8), Arg-Val-Tyr-Ile-His-Pro-Phe [SEQ ID NO:2]; AII(3-8), also known as desl-AIII or AIV, Val-Tyr-Ile-His-Pro-Phe [SEQ ID NO:3]; AII(1-7), Asp-Arg-Val-Tyr-Ile-His-Pro [SEQ ID NO:4]; AII(2-7). Arg-Val-Tyr-Ile-His-Pro [SEQ ID NO:5]; AII(3-7), Val-Tyr-Ile-His-Pro [SEQ ID NO:6]; AII(5-8), Ile-His-Pro-Phe [SEQ ID NO:7]; AII(1-6), Asp-Arg-Val-Tyr-Ile-His [SEQ ID NO:8]; AII(1-5), Asp-Arg-Val-Tyr-Ile [SEQ ID NO:9]; AII(1-4), Asp-Arg-Val-Tyr [SEQ ID NO:10]; and AII(1-3), Asp-Arg-Val [SEQ ID NO:11]. Other preferred embodiments include: Arg-norLeu-Tyr-Ile-His-Pro-Phe [SEQ ID NO:12] and Arg-Val-Tyr-norLeu-His-Pro-Phe [SEQ ID NO:13]. Still another preferred embodiment encompassed within the scope of the invention is a peptide having the sequence Asp-Arg-Pro-Tyr-Ile-His-Pro-Phe [SEQ ID NO:31]. AII(6-8), His-Pro-Phe [SEQ ID NO:14] and AII(4-8), Tyr-Ile-His-Pro-Phe [SEQ ID NO:15] were also tested and found not to be effective.

Another class of compounds of particular interest in accordance with the present invention are those of the general formula II

R²-R³-R⁴-R⁵-R⁶-R⁷-R⁸

in which R² is selected from the group consisting of H, Arg, Lys, Ala, Om, Citron, Ser(Ac), Sar, D-Arg and D-Lys;

R³ is selected from the group consisting of Val, Ala, Leu, norLeu, Ile, Gly, Pro, Aib, Acpc and Tyr;

R⁴ is selected from the group consisting of Tyr, Tyr(PO₃)₂, Thr, Ser, homoSer, azaTyr, and Ala;

R⁵ is selected from the group consisting of Ile, Ala, Leu, norLeu, Val and Gly;

R⁶ is His, Arg or 6-NH₂-Phe;

R⁷ is Pro or Ala; and

R⁸ is selected from the group consisting of Phe, Phe(Br), Ile and Tyr.

A particularly preferred subclass of the compounds of general formula II has the formula

R²-R³-Tyr-R⁵-His-Pro-Phe [SEQ ID NO:16]

wherein R^{2,} R³ and R⁵ are as previously defined. Particularly preferred is angiotensin III of the formula Arg-Val-Tyr-Ile-His-Pro-Phe [SEQ ID NO:2]. Other preferred compounds include peptides having the structures Arg-Val-Tyr-Gly-His-Pro-Phe [SEQ ID NO:17] and Arg-Val-Tyr-Ala-His-Pro-Phe [SEQ ID NO:18]. The fragment AII(4-8) was ineffective in repeated tests; this is believed to be due to the exposed tyrosine on the N-terminus.

In the above formulas, the standard three-letter abbreviations for amino acid residues are employed. In the absence of an indication to the contrary, the L-form of the amino acid is intended. Other residues are abbreviated as follows:

TABLE 1
Abbreviation for Amino Acids

| | |
|---|---|
| Me²Gly | N,N-dimethylglycyl |
| Bet | 1-carboxy-N,N,N-trimethylmethanaminium hydroxide inner salt |
| | (betaine) |
| Suc | Succinyl |
| Phe(Br) | p-bromo-L-phenylalanyl |
| azaTyr | aza-α′-homo-L-tyrosyl |
| Acpc | 1-aminocyclopentane carboxylic acid |
| Aib | 2-aminoisobutyric acid |
| Sar | N-methylglycyl (sarcosine) |
| Cit | Citron |
| Orn | Ornithine |

It has been suggested that AII and its analogues adopt either a gamma or a beta turn (Regoli, et al., Pharmacological Reviews 26:69 (1974). In general, it is believed that neutral side chains in position R³, R⁵ and R⁷ may be involved in maintaining the appropriate distance between active groups in positions R⁴, R⁶ and R⁸ primarily responsible for binding to receptors and/or intrinsic activity. Hydrophobic side chains in positions R³, R⁵ and R⁸ may also play an important role in the whole conformation of the peptide and/or contribute to the formation of a hypothetical hydrophobic pocket.

Appropriate side chains on the amino acid in position R² may contribute to affinity of the compounds for target receptors and/or play an important role in the conformation of the peptide. For this reason, Arg and Lys are particularly preferred as R². Alternatively, R₂ may be H, Ala, Orn, Citron, Ser(Ac), Sar, D-Arg, or D-Lys.

For purposes of the present invention, it is believed that R³ may be involved in the formation of linear or nonlinear hydrogen bonds with R⁵ (in the gamma turn model) or R⁶ (in the beta turn model). R³ would also participate in the first turn in a beta antiparallel structure (which has also been proposed as a possible structure). In contrast to other positions in general formula I, it appears that beta and gamma branching are equally effective in this position. Moreover, a single hydrogen bond may be sufficient to maintain a relatively stable conformation. Accordingly, R³ may suitably be selected from Lys, Val, Ala, Leu, norLeu, Ile, Gly, Pro, Aib, Acpc and Tyr.

With respect to R⁴, conformational analyses have suggested that the side chain in this position (as well as in R³ and R⁵) contribute to a hydrophobic cluster believed to be essential for occupation and stimulation of receptors. Thus, R⁴ is preferably selected from Tyr, Thr, Tyr (PO₃)₂, homoSer, Ser and azaTyr. In this position, Tyr is particularly preferred as it may form a hydrogen bond with the receptor site capable of accepting a hydrogen from the phenolic hydroxyl (Regoli, et al. (1974), supra). It has also been found that R⁴ can be Ala.

In position R⁵, an amino acid with a β aliphatic or alicyclic chain is particularly desirable. Therefore, while Gly is suitable in position R⁵, it is preferred that the amino acid in this position be selected from Ile, Ala, Leu, norLeu, and Val.

In the angiotensinogen, AI, AI analogues, AI fragments and analogues thereof, AII analogues, fragments and analogues of fragments of particular interest in accordance with the present invention, R⁶ is His, Arg or 6-NH₂-Phe. The unique properties of the imidazole ring of histidine (e.g., ionization at physiological pH, ability to act as proton donor or acceptor, aromatic character) are believed to contribute to its particular utility as R⁶. For example, conformational models suggest that His may participate in hydrogen bond formation (in the beta model) or in the second turn of the antiparallel structure by influencing the orientation of R⁷. Similarly, it is presently considered that R⁷ should be Pro or Ala in order to provide the most desirable orientation of R⁸. In position R8, both a hydrophobic ring and an anionic carboxyl terminal appear to be particularly useful in binding of the analogues of interest to receptors; therefore, Tyr, Ile, Phe(Br), and especially Phe are preferred for purposes of the present invention.

Analogues of particular interest include the following:

TABLE 2

Angiotensin II Analogues

| | | |
|---|---|---|
| All Analogue | Amino Acid Sequence | Sequence Identifier |

| | | |
|---|---|---|
| Analogue 1 | Asp-Arg-Val-Tyr-Val-His-Pro-Phe | SEQ ID NO:19 |
| Analogue 2 | Asn-Arg-Val-Tyr-Val-His-Pro-Phe | SEQ ID NO:20 |
| Analogue 3 | Ala-Pro-Gly-Asp-Arg-Ile-Tyr-Val-His-Pro-Phe | SEQ ID NO:21 |
| Analogue 4 | Glu-Arg-Val-Tyr-Ile-His-Pro-Phe | SEQ ID NO:22 |
| Analogue 5 | Asp-Lys-Val-Tyr-Ile-His-Pro-Phe | SEQ ID NO:23 |
| Analogue 6 | Asp-Arg-Ala-Tyr-Ile-His-Pro-Phe | SEQ ID NO:24 |
| Analogue 7 | Asp-Arg-Val-Thr-Ile-His-Pro-Phe | SEQ ID NO:25 |
| Analogue 8 | Asp-Arg-Val-Tyr-Leu-His-Pro-Phe | SEQ ID NO:26 |
| Analogue 9 | Asp-Arg-Val-Tyr-Ile-Arg-Pro-Phe | SEQ ID NO:27 |
| Analogue 10 | Asp-Arg-Val-Tyr-Ile-His-Ala-Phe | SEQ ID NO:28 |
| Analogue 11 | Asp-Arg-Val-Tyr-Ile-His-Pro-Tyr | SEQ ID NO:29 |
| Analogue 12 | Pro-Arg-Val-Tyr-Ile-His-Pro-Phe | SEQ ID NO:30 |
| Analogue 13 | Asp-Arg-Pro-Tyr-Ile-His-Pro-Phe | SEQ ID NO:31 |
| Analogue 14 | Asp-Arg-Val-Tyr(PO₃)₂-Ile-His-Pro-Phe | SEQ ID NO:32 |
| Analogue 15 | Asp-Arg-norLeu-Tyr-Ile-His-Pro-Phe | SEQ ID NO:33 |
| Analogue 16 | Asp-Arg-Val-Tyr-norLeu-His-Pro-Phe | SEQ ID NO:34 |
| Analogue 17 | Asp-Arg-Val-homoSer-Tyr-Ile-His-Pro-Phe | SEQ ID NO:35 |

Other particularly preferred embodiments of the active agents include:

| | | | |
|---|---|---|---|
| 1GD | Ala4-AII(1-7) | DRVAIHP | SEQ ID NO:38 |
| 2GD | Pro3-AII(1-7) | DRPYIHP | SEQ ID NO:39 |
| 5GD | Lys3-AII(1-7) | DRKYIHP | SEQ ID NO:40 |
| 9GD | NorLeu-AII(1-7) | DR(nor)YIHP | SEQ ID NO:41 |
| GSD | Ile⁸-AII | DRVYIHPI | SEQ ID NO:42 |
| 28 | | | |
| | Ala3aminoPhe6 | DRAYIF*PF | SEQ ID NO:43 |
| | AIII: | | |
| | Ala3-AIII | RVAIHPF | SEQ ID NO:44 |
| | Gly¹-AII | GRVYIHPF | SEQ ID NO:45 |
| | NorLeu⁴-AIII | --RVYnLHPF | SEQ ID NO:46 |
| | Acpc³-AII | DR(Acpc)YIHPF | SEQ ID NO:47 |
| GSD | Orn²-AII | D(Orn)VYIHPF | SEQ ID NO:48 |
| 37B | | | |
| GSD | Citron²-AII | D(Citron)VYIHPF | SEQ ID NO:49 |
| 38B | | | |
| 3GD | Pro³Ala⁴-AII(1-7) | DRPAIHP | SEQ ID NO:50 |

The polypeptides of the instant invention may be synthesized by any conventional method, including, but not limited to, those set forth in J. M. Stewart and J. D. Young, Solid Phase Peptide Synthesis, 2nd ed., Pierce Chemical Co., Rockford, III. (1984) and J. Meienhofer, Hormonal Proteins and Peptides, Vol. 2, Academic Press, New York, (1973) for solid phase synthesis and E. Schroder and K. Lubke, The Peptides, Vol. 1, Academic Press, New York, (1965) for solution synthesis. The disclosures of the foregoing treatises are incorporated by reference herein.

In general, these methods involve the sequential addition of protected amino acids to a growing peptide chain (U.S. Pat. No. 5,693,616, herein incorporated by reference in its entirety). Normally, either the amino or carboxyl group of the first amino acid and any reactive side chain group are protected. This protected amino acid is then either attached to an inert solid support, or utilized in solution, and the next amino acid in the sequence, also suitably protected, is added under conditions amenable to formation of the amide linkage. After all the desired amino acids have been linked in the proper sequence, protecting groups and any solid support are removed to afford the crude polypeptide. The polypeptide is desalted and purified, preferably chromatographically, to yield the final product.

Preferably, peptides are synthesized according to standard solid-phase methodologies, such as may be performed on an Applied Biosystems Model 430A peptide synthesizer (Applied Biosystems, Foster City, Calif.), according to manufacturer's instructions. Other methods of synthesizing peptides or peptidomimetics, either by solid phase methodologies or in liquid phase, are well known to those skilled in the art. Alternatively, the peptides can be produced by standard molecular biological techniques.

In one aspect of the present invention, in vitro, ex vivo, and in vivo methods for promoting dendritic cell proliferation or differentiation, comprising contacting the dendritic cells with an amount effective to promote dendritic cell proliferation or differentiation of one or more of the active agents of the invention, and/or the DNA or RNA encoding any of these, either alone, combined, or in further combination with other compounds for promoting dendritic cell proliferation, differentiation, and/or tumor vaccine production or efficacy, including but not limited to cytokines, cytokine-encoding nucleic acid molecules, polycations (such as polylysine), heat shock proteins, viral-like particles, and incomplete Freund's adjuvant (IFA). When the active agents are added in combination with other active agents or with other compounds, such combinations can include adding compounds simultaneously or sequentially, or preparing fusions between the compounds.

In a preferred embodiment, the method is used to promote tumor vaccine production. In a more preferred embodiment, the methods are used to promote the production of a cell-based tumor vaccine. In one example, hematopoietic progenitor cells are isolated from a cancer patient and are contacted with one or more active agents of the invention, alone or in combination with one or more cytokines, such as GM-CSF and Flt3-L to promote differentiation into dendritic cells, and to promote proliferation of dendritic cells. Alternatively, dendritic cells are isolated from the cancer patient and contacted with one or more active agents of the invention, alone or in combination with one or more cytokines, such as GM-CSF and Flt3-L, to promote proliferation of dendritic cells. The dendritic cells are then either pulsed with TAA peptides or proteins, transduced with genes coding for TAAs, pulsed with tumor extracts or tumor-derived RNAs, or fused with tumor cells, in the presence of one or more of the active agents of the invention, before replacement into the patient.

In another embodiment of this aspect of the method a tumor cell is engineered, either derived from the cancer patient or from tumor cell lines, using standard molecular biology techniques, to express one or more active agents of the invention, either alone or in combination with a cytokine that stimulates the differentiation of dendritic cells, such as GM-CSF, Flt3-L, and/or one or more of the active agents of the invention. The goal is to create very high concentrations of the active agent local to the injected tumor cells, in order to produce a high concentration of dendritic cells for presenting TAA to prime helper and killer T cells in vivo. In a variation of this embodiment, the active agent(s) is/are coated on, or encapsulated within, a carrier, such as a cell-sized polymer microsphere which are administered to the patient in the vicinity of the tumor.

In a further embodiment of this aspect, the present invention provides vector-based vaccines for promoting tumor vaccine production, comprising administering to a patient in need thereof a vector encoding a TAA and/or a cytokine in combination with one or more of the active agents of the invention operably linked to a promoter for driving expression of the active agent. Vectors encoding TAAs and/or cytokines have been described in the literature (Chen and Wu (1998); Gouttefangeas and Rammensee (2000)); such vectors include but are not limited to adenovirus vectors, vaccinia virus vectors, bacterial vectors, and combinations thereof.

Other embodiments include administering the active agent in combination with peptide-based vaccines (such as antigenic determinants from TAAs), protein-based vaccines (thus eliminating the potential problems associated with vector-based delivery of TAAs and cytokines), and DNA-based vaccines (see, for example, Cho et al., Nature Biotechnology 18:509-514 (2000)).

For use in the present invention, the active agents may be made up in a solid form (including granules, powders or suppositories) or in a liquid form (e.g., solutions, suspensions, or emulsions), and may be subjected to conventional pharmaceutical operations such as sterilization and/or may contain conventional adjuvants, such as stabilizers, wetting agents, emulsifiers, preservatives, cosolvents, suspending agents, viscosity enhancing agents, ionic strength and osmolality adjustors and other excipients in addition to buffering agents. Suitable water soluble preservatives which may be employed in the drug delivery vehicle include sodium bisulfite, sodium thiosulfate, ascorbate, benzalkonium chloride, chlorobutanol, thimerosal, phenylmercuric borate, parabens, benzyl alcohol, phenylethanol or antioxidants such as Vitamin E and tocopherol and chelators such as EDTA and EGTA. These agents may be present, generally, in amounts of about 0.001% to about 5% by weight and, preferably, in the amount of about 0.01 to about 2% by weight.

For administration, the active agents are ordinarily combined with one or more adjuvants appropriate for the indicated route of administration. The compounds may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and tableted or encapsulated for conventional administration. Alternatively, the compounds of this invention may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

For use herein, the active agents may be administered by any suitable route, including local delivery, parentally, transdermally, or topically in dosage unit formulations containing conventional pharmaceutically acceptable carriers, adjuvants, and vehicles. The term parenteral as used herein includes, subcutaneous, intravenous, intramuscular, intrasternal, intratendinous, intraspinal, intracranial, intrathoracic, infusion techniques or intraperitoneally.

In a preferred embodiment, the active agents in combination with cell-based, vector-based, peptide-based, protein-based, and DNA-based vaccines are administered to a patient in need thereof by intramuscular or intradermal injection. Viral vector-based and DNA-based vaccines can also be administered to the host via a gene gun. In another preferred embodiment, administration is subcutaneous, intravenous, or direct injection into or in the area surrounding the tumor or after in vitro or ex vivo production of the tumor vaccine.

For topical administration, the active agents in combination with the vaccines may be formulated as is known in the art for direct application to a target area containing a tumor. Conventional forms for this purpose include patches, and aerosols. The percent by weight of the active agent of the invention present in a topical formulation will depend on various factors, but generally will be from 0.005% to 95% of the total weight of the formulation, and typically 1-25% by weight.

Suppositories for rectal administration of the active agents in combination with the vaccines can be prepared by mixing the drug with a suitable non-irritating excipient such as cocoa butter and polyethylene glycols which are solid at ordinary temperatures, but liquid at the rectal temperature and will therefore melt in the rectum and release the drug.

Solid dosage forms for oral administration may include capsules, tablets, pills, powders and granules. In such solid dosage forms, the active compound may be admixed with at least one inert diluent such as sucrose, lactose or starch. Such dosage forms may also comprise, as is normal practice, additional substances other than inert diluents, e.g., lubricating agents such as magnesium stearate. In the case of capsules, tablets and pills, the dosage forms may also comprise buffering agents. Tablets and pills can additionally be prepared with enteric coatings.

Liquid dosage forms for oral administration may include pharmaceutically acceptable emulsions, solutions, suspensions, syrups and elixirs containing inert duluents commonly used in the art, such as water. Such compositions may also comprise adjuvants, such as wetting agents, emlusifying and suspending agents and sweetening, flavoring and perfuming agents.

The dosage and treatment regimen for promoting tumor vaccine production with the active agents is based on a variety of factors, including the age, weight, sex, medical condition of the individual, the severity of the condition, the route of administration, and the particular compound employed. Thus, the dosage regimen may vary widely, but can be determined by a physician using standard methods. Dosage levels of the order of between 0.1 ng/kg and 10 mg/kg of the active agents per body weight are useful for all methods of use disclosed herein. For example, promotion of tumor vaccine production using the active agents can be accomplished by topical application of the composition to the affected areas containing the tumor two or three times a day for as long as is needed.

In a further aspect, the present invention provides kits for promoting tumor vaccine production, wherein the kits comprise an effective amount of the active agents of the invention to promote tumor vaccine production, and instructions for using the amount effective of active agent to promote tumor vaccine production. In a preferred embodiment, the kits also contain an amount effective to promote tumor vaccine production of one or more other compounds, including but not limited to cytokines, cytokine-encoding nucleic acid molecules, polycations (such as polylysine), heat shock proteins, viral-like particles, and incomplete Freund's adjuvant (IFA). Effective dosages of the active agents of the invention to promote tumor vaccine production or improve the efficacy of a tumor vaccine are between about 0.1 ng/kg and 10 mg/kg, as discussed above.

In another aspect of the invention, pharmaceutical compositions are provided that comprise an amount effective to promote tumor vaccine production of one or more of the active agents of the invention in combination with an amount effective to promote tumor vaccine production of cytokines, cytokine-encoding nucleic acid molecules, polycations (such as polylysine), heat shock proteins, viral-like particles, and incomplete Freund's adjuvant (IFA).

### EXAMPLES

1. Effect of Angiotensin Peptides on Dendritic Cell Proliferation

Isolation of Dendritic Cells from Mice Treated with flt3 Ligand: Spleens were enriched in vivo with dendritic cells by stimulation with flt3 ligand (Marakovsky et al, 1996). C57B1/6 mice were subcutaneously injected with 4×10⁶ flt3 ligand-secreting B16 melanoma cells prepared by transfection of murine flt3 ligand cDNA with the retroviral vector MFG. Spleen cells were harvested after the tumors reached 2-3 cm in diameter, at which time the spleens were 5-10 fold enlarged over those of untreated mice.

The spleens were harvested and the cells were dispersed through sterile screens. A single cell suspension of whole splenocytes was resuspended in a high-density BSA solution (1 ml/spleen) containing 10.6 g BSA, 18.6 ml PBS, 2.9 ml 1 N NaOH, and 6.5 ml water. After overlaying 2 ml of ice-cold RPMI, the splenocyte/BSA solution was centrifuged for 15 minutes at 9500 g. About 5×10⁷ dendritic cells/spleen were recovered from the interface and resuspended in RPMI. The cells were characterized by FACS using anti-B220, CD11c, CD11b, CD3, CD4, CD8, CD86, CD80, GR-1 and I-A^{b} antibodies. The surface phenotype of these cells was similar to dendritic cells generated by injection of recombinant murine flt3 ligand. This dendritic cell populations is not homogenous with regards to its myeloid and/or lymphoid phenotype, but it displays a uniform MHC class II surface expression and robust antigen presenting capability.

Culture of Dendritic Cells: Twenty four hours after isolation, these cells were counted and placed in culture in RPMI 1640 supplemented with 10% fetal calf serum, 2 mM 1 glutamine, 1 mM sodium pyruvate, 50 μM mercaptoethanol, 100 U/ml penicillin and 100 μg/ml streptomycin at a concentration of 1×10³ cells/well of a 96 well plate. One third of the wells were counted at random to ensure even distribution of the cells to the wells. Various concentrations of angiotensin peptides (1-100 μg/ml) were added to the cultures in duplicate. These cultures were done in the presence of various cytokines and cytokine combinations as follow: (1) medium alone; (2) 25 ng/ml murine recombinant Interleukin 6 (IL-6); (3) 50 ng/ml murine recombinant stem cell factor (SCF); (4) 25 ng/ml IL-6+50 ng/ml murine recombinant Flt3 ligand+50 ng/ml murine recombinant granulocyte macrophage-colony stimulating factor (GM-CSF) and (5) 50 ng/ml GM-CSF. The plates were then cultured at 37° C. in 5% CO₂ in humidified air. At days 1, 4 and 7 after addition of the cytokines and angiotensin peptides to the cultures, the number of cells per well was ascertained by phase contrast microscopy.

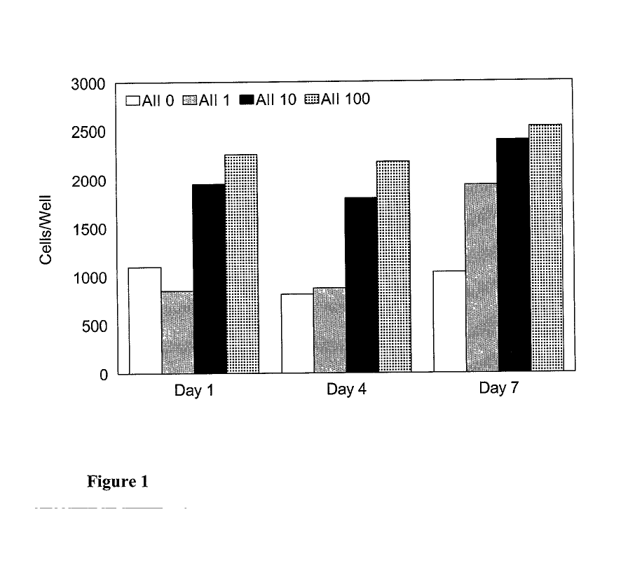

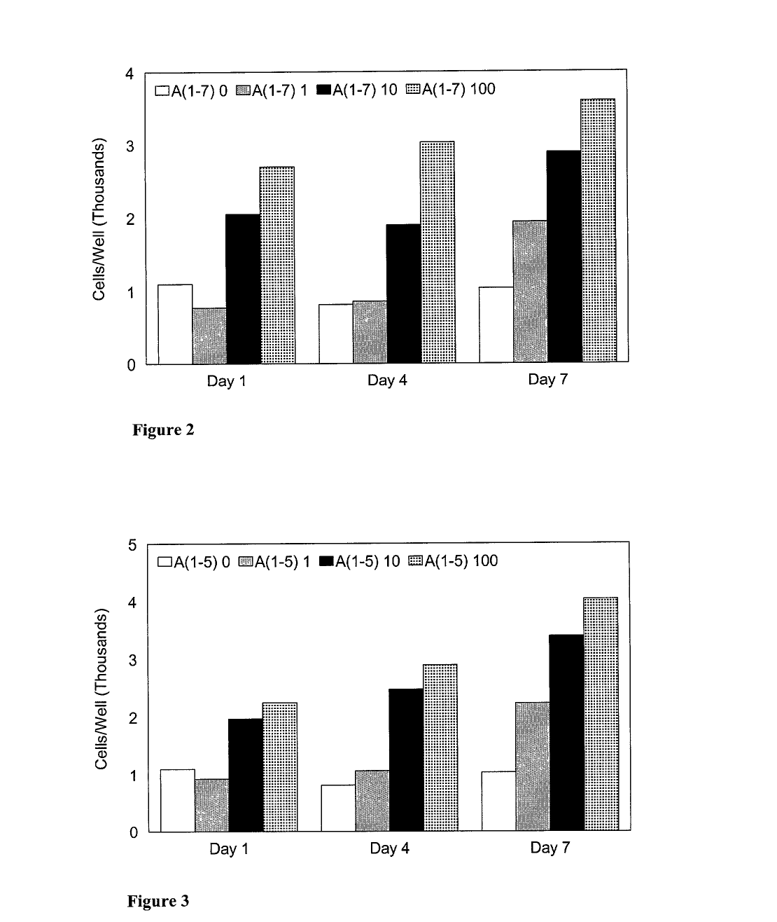

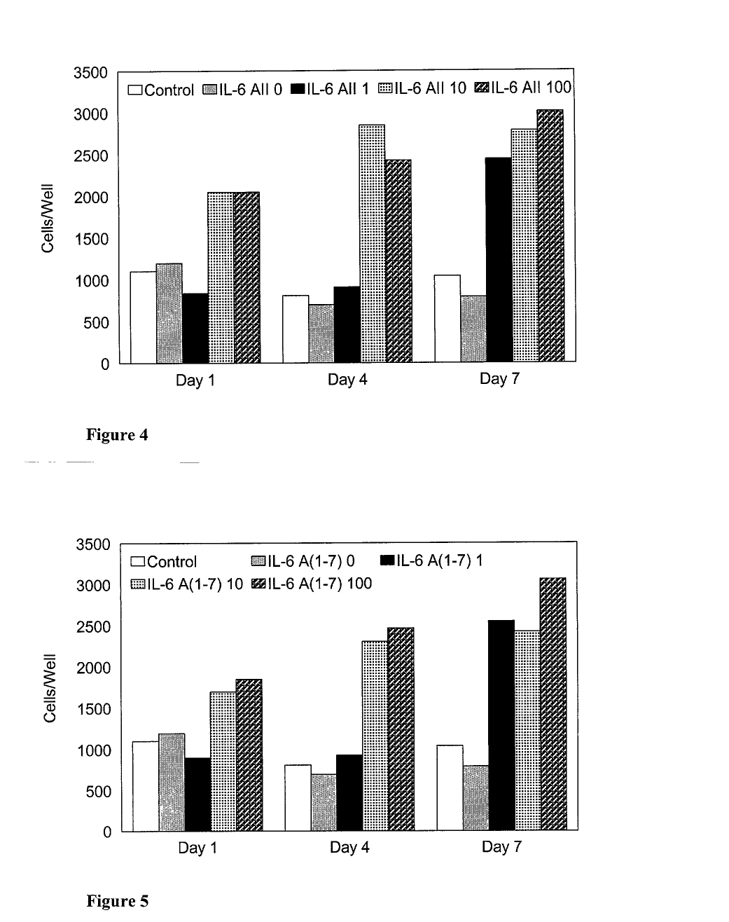

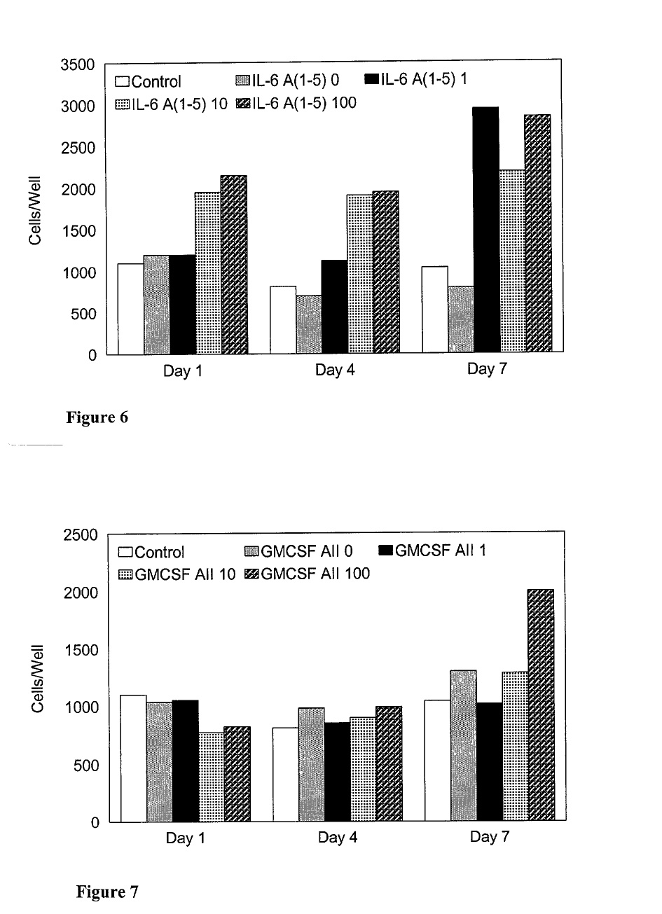

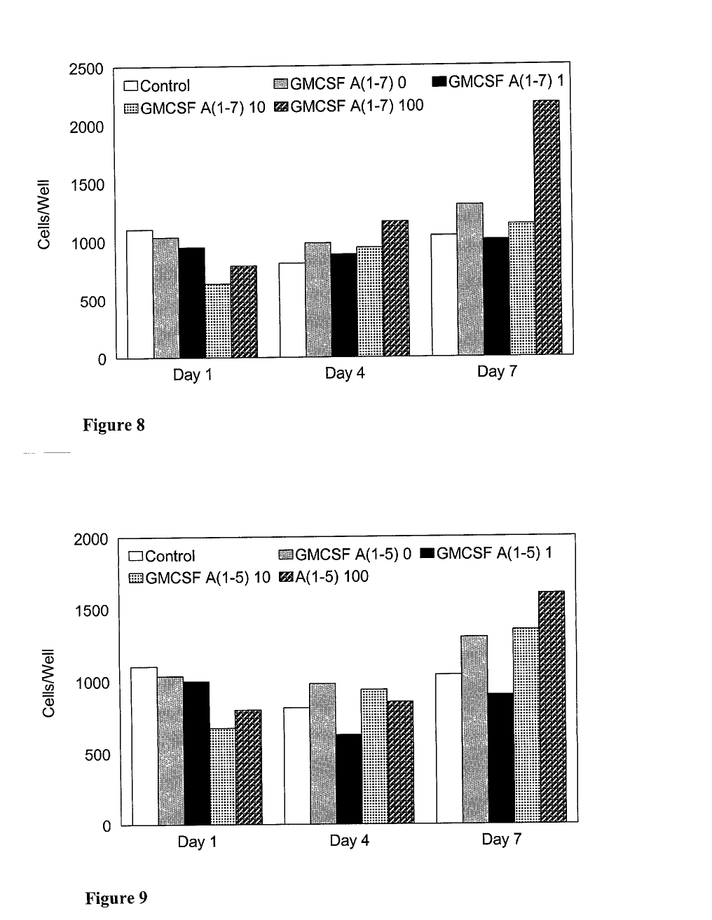

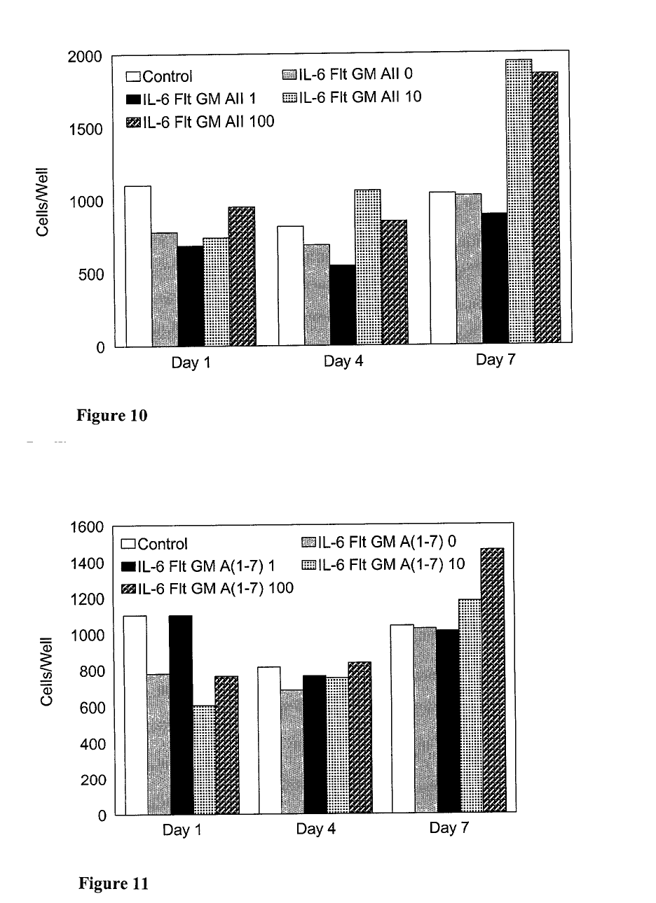

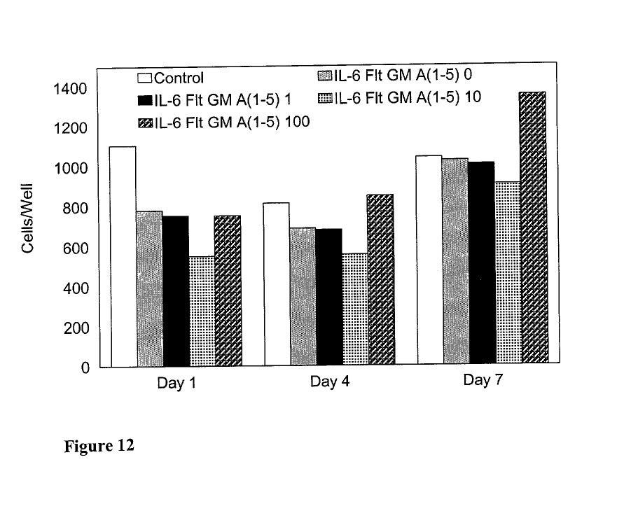

Results: Addition of angiotensin peptides to cultures with medium alone resulted in a concentration and time dependent increase in dendritic cell number per well (FIGS. 1-3). Addition of IL-6 to the cultures did not affect the number of cells per well, however, addition of angiotensin peptides in combination with IL-6 resulted in a time and concentration dependent increase in cell number (FIGS. 4-6). Exposure of these dendritic cells to GM-CSF or GM-CSF in combination with IL-6 and Flt 3 ligand did not increase cellular proliferation in these cultures and angiotensin peptides increased cell number at higher concentrations and later time points (FIGS. 7-12). These data show that the exposure of dendritic cells to angiotensin peptides increases the proliferation of these cells. Further, the peptides continue to increase the proliferation of dendritic cells in the presence of various cytokines, alone or in combination. Since dendritic cells are integral to the formation of tumor vaccines, as the cells responsible for presentation of tumor antigens in the generation of an immune response, these data support the hypothesis that these peptides increase the formation of tumor vaccines.

### Example 2.

Effect of Angiotensin Peptides on Ability of Dendritic Cells to Present Antigen

Isolation of Dendritic Cells: Performed as above for example 1.

Culture of Dendritic Cells: Twenty four hours after isolation, these cells were counted and placed in culture in RPMI 1640 supplemented with 10% fetal calf serum, 2 mM 1 glutamine, 1 mM sodium pyruvate, 50 μM mercaptoethanol, 100 U/ml penicillin and 100 μg/ml streptomycin at a concentration of 1.5×10³ cells/well of a 96 well plate. After aliquoting into the 96 well plates, the cells were irradiated with 1400 cGy with a cesium irradiator to prevent dendritic cell proliferation. After irradiation, various concentrations of angiotensin peptides (1-100 μg/ml) were added to the cultures in triplicate. These cultures were done with and without 25 ng/ml murine recombinant Interleukin 6 (IL-6). The plates were then cultured at 37° C. in 5% CO₂ in humidified air. At 4 days after addition of the angiotensin peptides to the cultures, the ability of the dendritic cells to present antigen was assessed.

Antigen Presentation: A hybridoma specific for the OVA/K^{b} ligand, B3Z, was the kind gift of Dr. Nilabh Shastri at the University of California, Berkeley. Further, these cells were produced by fusion with a lacZ-inducible derivative of BW5147. (Karttunen et al., PNAS 89:6020-6024, 1992) The resultant hybridoma allows detection of T cell activation in an antigen-specific manner through the evaluation of lacZ activity.

After 4 days in culture, the dendritic cells were washed and either medium or 1 μg/ml of a fragment of ovalbumin, Ser-Ile-Ile-Asn-Phe-Glu-Lys-Leu (OVA, American Peptide Company, Sunnyvale, Calif.) (hereinafter “antigen”), to which the T cell hybridoma utilized in this study is responsive. The dendritic cells were pulsed with antigen for 6 hours at 37° C. in 5% CO₂ in humidified air and then washed to remove antigen not associated with dendritic cells. B3Z T cell hybridoma cells, 1.2×10⁴ per well, were added to the antigen pulsed dendritic cells and incubated at 37° C. in 5% CO₂ in humidified air overnight. The cells were then centrifuged and washed twice with phosphate buffered saline, pH 7.4, containing 2% fetal bovine serum and 10 mM HEPES. Thereafter, lacZ activity was measured using ImaGene Green (Molecular Probes, Eugene, OR) as a substrate. The substrate was added to the cells, placed at 4° C. for one hour and the reaction was stopped with phenylethyl-β-D-thiogalactopyranoside. The amount of fluorescence produced (proportional to lacZ activity as a measure of T cell activity through antigen presention) was evaluated using a multiwell plate reader that measures fluorescence.

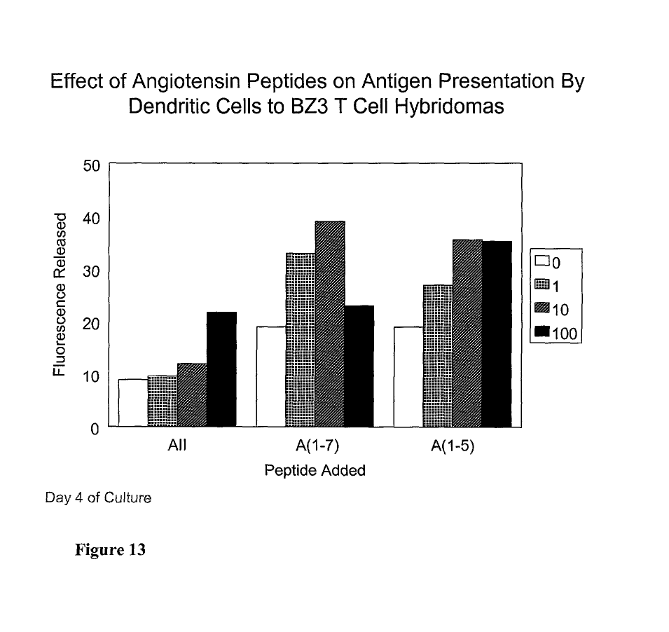

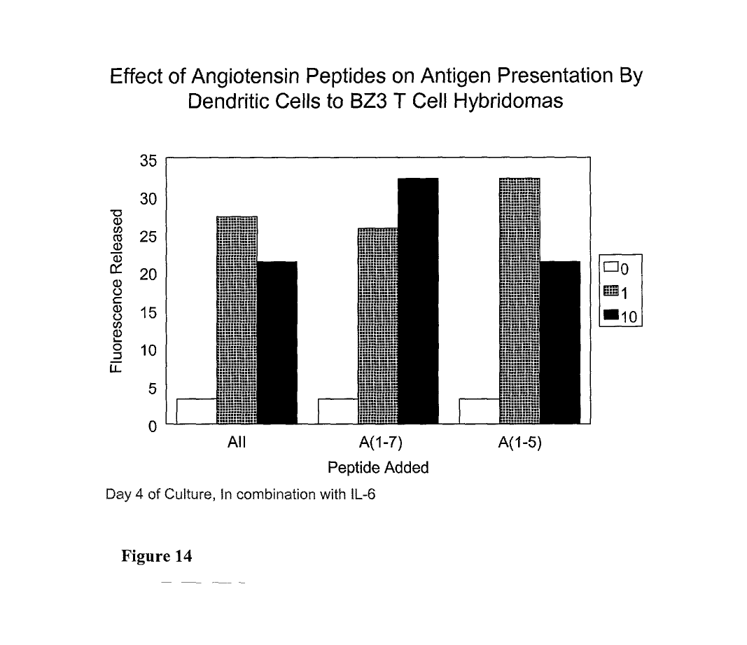

Results: The results, presented in FIGS. 13-14, demonstrate that exposure of the dendritic cells to angiotensin peptides, either in the presence or absence of IL-6, increased the ability of the cells to present antigen. These data further support the use of the peptides of the present invention to promote dendritic cell activity, and thus to promote tumor vaccine production.

We claim

1. A method for promoting dendritic cell proliferation or differentiation comprising contacting the dendritic cells with an amount effective to promote dendritic cell proliferation or differentiation of at least one active agent comprising a sequence of at least three contiguous amino acids of groups R¹-R⁸ in the sequence of general formula I
R¹-R²-R³-R⁴-R⁵-R⁶-R⁷-R⁸
wherein R¹ s selected from the group consisting of H, Asp, Glu, Asn, Acpc (1-aminocyclopentane carboxylic acid), Ala, Me²Gly, Pro, Bet, Glu(NH₂), Gly, Asp(NH₂) and Suc,
R^{B} is selected from the group consisting of Arg, Lys, Ala, Om, Ser(Ac), Sar, D-Arg and D-Lys;
R³ is selected from the group consisting of Val, Ala, Leu, Lys, norLeu, Ile, Gly, Pro, Aib, Acpc and Tyr;
R⁴ is selected from the group consisting of Tyr, Tyr(PO₃)₂, Thr, Ser, Ala, homoSer and azaTyr;
R⁵ is selected from the group consisting of Ile, Ala, Leu, norLeu, Val and Gly;
R⁶ is selected from the group consisting of His, Arg or 6-NH₂-Phe;
R⁷ is selected from the group consisting of Pro or Ala; and
R⁸ is selected from the group consisting of phe, phe(br), ile and tyr, excluding sequences including r⁴ as a terminal tyr group:

2. The method of claim 1 wherein the active agent comprises a sequence of at least four contiguous amino acids of groups R¹-R⁸ in the sequence of general formula I.

3. The method of claim 1 wherein the active agent comprises a sequence of at least five contiguous amino acids of groups R^{1-R}⁸ in the sequence of general formula I.

4. The method of claim 1 wherein the active agent comprises a sequence of at least six contiguous amino acids of groups R¹-R⁸ in the sequence of general formula I.

5. The method of claim 1 wherein the active agent comprises a sequence of at least seven contiguous amino acids of groups R^{1-R}⁸ in the sequence of general formula I.

6. The method of claim 1 wherein the active agent consists essentially of a sequence of at least three contiguous amino acids of groups R¹-R⁸ in the sequence of general formula I.

7. The method of claim 1 wherein the active agent consists essentially of a sequence of at least four contiguous amino acids of groups R¹-R⁸ in the sequence of general formula I.

8. The method of claim 1 wherein the active agent consists essentially of a sequence of at least five contiguous amino acids of groups R¹-R⁸ in the sequence of general formula I.

9. The method of claim 1 wherein the active agent consists essentially of a sequence of at least six contiguous amino acids of groups R¹-R⁸ in the sequence of general formula I.

10. The method of claim 1 wherein the active agent consists essentially of a sequence of at least seven contiguous amino acids of groups R^{1-R}⁸ in the sequence of general formula I.

11. The method of claim 1 wherein the active agent comprises a sequence selected from the group consisting of angiotensinogen, SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ
ID NO:9, SEQ ID NO:10, SEQ ID NO:11, SEQ ID NO:12, SEQ ID NO:13, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO: 32, SEQ ID NO:33, SEQ ID NO: 34; SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, and SEQ ID NO:50.

12. The method of claim 1 wherein the active agent comprises a sequence selected from the group consisting of SEQ ID NO:1, SEQ ID NO:4 and SEQ ID NO:9.

13. The method of claim 12 wherein the active agent comprises the sequence of SEQ ID NO:4.

14. The method of claim 1 wherein the active agent consists essentially of a sequence selected from the group consisting of angiotensinogen, SEQ ID NO:1, SEQ ID NO:2, SEQ ID NO:3, SEQ ID NO:4, SEQ ID NO:5, SEQ ID NO:6, SEQ ID NO:7, SEQ ID
NO:8, SEQ ID NO:9, SEQ ID NO:10, SEQ ID NO:11, SEQ ID NO:12, SEQ ID NO:13, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO: 32, SEQ ID NO:33, SEQ ID NO: 34; SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, and SEQ ID NO:50.

15. The method of claim 1 wherein the active agent consists essentially of a sequence selected from the group consisting of SEQ ID NO: 1, SEQ ID NO:4 or SEQ ID NO:9.

16. The method of claim 15 wherein the active agent consists essentially of the sequence selected of SEQ ID NO:4.

17. The method of claim 15 wherein the method is used to promote tumor vaccine production.

18. The method of claim 16 wherein the method is used to promote tumor vaccine production.

19. A pharmaceutical composition comprising:
(a) an amount effective to promote dendritic cell proliferation of an active agent consisting essentially of a sequence selected from the group consisting of SEQ ID NO: 1, SEQ ID NO:4 or SEQ ID NO:9;
(b) an amount effective to promote dendritic cell proliferation or tumor vaccine production cytokine selected from the group consisting of GM-CSF, M-CSF, TNF-c, IL-1, I1-2, IL-4, IL-6, IL-12, Flt3-L, interferons, immunoglobulin superfamily molecules, chemokines; and
(c) a pharmaceutically acceptable carrier.


SEQUENCE LISTING (50 sequences)

SEQ ID NO: 1 - protein, 8 aa, Artificial Sequence
  Description of Artificial SequenceAII
DRVYIHPF

SEQ ID NO: 2 - protein, 7 aa, Artificial Sequence
  Description of Artificial SequenceAII (2-8)
RVYIHPF

SEQ ID NO: 3 - protein, 6 aa, Artificial Sequence
  Description of Artificial SequenceAII (3-8)
VYIHPF

SEQ ID NO: 4 - protein, 7 aa, Artificial Sequence
  Description of Artificial SequenceAII (1-7)
DRVYIHP

SEQ ID NO: 5 - protein, 6 aa, Artificial Sequence
  Description of Artificial SequenceAII (2-7)
RVYIHP

SEQ ID NO: 6 - protein, 5 aa, Artificial Sequence
  Description of Artificial SequenceAII (3-7)
VYIHP

SEQ ID NO: 7 - protein, 4 aa, Artificial Sequence
  Description of Artificial SequenceAII (5-8)
IHPF

SEQ ID NO: 8 - protein, 6 aa, Artificial Sequence
  Description of Artificial SequenceAII (1-6)
DRVYIH

SEQ ID NO: 9 - protein, 5 aa, Artificial Sequence
  Description of Artificial SequenceAII (1-5)
DRVYI

SEQ ID NO: 10 - protein, 4 aa, Artificial Sequence
  Description of Artificial SequenceAII (1-4)
DRVY

SEQ ID NO: 11 - protein, 3 aa, Artificial Sequence
  Description of Artificial SequenceAII (1-3)
DRV

SEQ ID NO: 12 - protein, 7 aa, Artificial Sequence
  Description of Artificial SequenceAII analogue
RXYIHPF

SEQ ID NO: 13 - protein, 7 aa, Artificial Sequence
  Description of Artificial SequenceAII analogue
RVYXHPF

SEQ ID NO: 14 - protein, 3 aa, Artificial Sequence
  Description of Artificial SequenceAII (6-8)
HPF

SEQ ID NO: 15 - protein, 5 aa, Artificial Sequence
  Description of Artificial SequenceAII (4-8)
YIHPF

SEQ ID NO: 16 - protein, 7 aa, Artificial Sequence
  Description of Artificial SequenceAII analogue class
XXYXHPF

SEQ ID NO: 17 - protein, 7 aa, Artificial Sequence
  Description of Artificial SequenceAII analogue
RVYGHPF

SEQ ID NO: 18 - protein, 7 aa, Artificial Sequence
  Description of Artificial SequenceAII analogue
RVYAHPF

SEQ ID NO: 19 - protein, 8 aa, Artificial Sequence
  Description of Artificial SequenceAII analogue 1
DRVYVHPF

SEQ ID NO: 20 - protein, 8 aa, Artificial Sequence
  Description of Artificial SequenceAII analogue 2
NRVYVHPF

SEQ ID NO: 21 - protein, 11 aa, Artificial Sequence
  Description of Artificial SequenceAII analogue 3
APGDRIYVHPF

SEQ ID NO: 22 - protein, 8 aa, Artificial Sequence
  Description of Artificial SequenceAII analogue 4
ERVYIHPF

SEQ ID NO: 23 - protein, 8 aa, Artificial Sequence
  Description of Artificial SequenceAII analogue 5
DKVYIHPF

SEQ ID NO: 24 - protein, 8 aa, Artificial Sequence
  Description of Artificial SequenceAII analogue 6
DRAYIHPF

SEQ ID NO: 25 - protein, 8 aa, Artificial Sequence
  Description of Artificial SequenceAII analogue 7
DRVTIHPF

SEQ ID NO: 26 - protein, 8 aa, Artificial Sequence
  Description of Artificial SequenceAII analogue 8
DRVYLHPF

SEQ ID NO: 27 - protein, 8 aa, Artificial Sequence
  Description of Artificial SequenceAII analogue 9
DRVYIRPF

SEQ ID NO: 28 - protein, 8 aa, Artificial Sequence
  Description of Artificial SequenceAII analogue 10
DRVYIHAF

SEQ ID NO: 29 - protein, 8 aa, Artificial Sequence
  Description of Artificial SequenceAII analogue 11
DRVYIHPY

SEQ ID NO: 30 - protein, 8 aa, Artificial Sequence
  Description of Artificial SequenceAII analogue 12
PRVYIHPF

SEQ ID NO: 31 - protein, 8 aa, Artificial Sequence
  Description of Artificial SequenceAII analogue 13
DRPYIHPF

SEQ ID NO: 32 - protein, 8 aa, Artificial Sequence
  Description of Artificial SequenceAII analogue 14
DRVYIHPF

SEQ ID NO: 33 - protein, 8 aa, Artificial Sequence
  Description of Artificial SequenceAII analogue 15
DRXYIHPF

SEQ ID NO: 34 - protein, 8 aa, Artificial Sequence
  Description of Artificial SequenceAII analogue 16
DRVYXHPF

SEQ ID NO: 35 - protein, 9 aa, Artificial Sequence
  Description of Artificial SequenceAII analogue 17
DRVSYIHPF

SEQ ID NO: 36 - protein, 8 aa, Artificial Sequence
  Description of Artificial Sequencep-aminophenylalanine 6 AII
DRVYIXPF

SEQ ID NO: 37 - protein, 10 aa, Artificial Sequence
  Description of Artificial Sequence angiotensin I
DRVYIHPFHL

SEQ ID NO: 38 - protein, 7 aa, Artificial Sequence
  Description of Artificial Sequence 1GDAla4-AII(1-7)
DRVAIHP

SEQ ID NO: 39 - protein, 7 aa, Artificial Sequence
  Description of Artificial Sequence 2GD Pro3-AII(1-7)
DRPYIHP

SEQ ID NO: 40 - protein, 7 aa, Artificial Sequence
  Description of Artificial Sequence 5GD Lys 3-AII(1-7)
DRKYIHP

SEQ ID NO: 41 - protein, 7 aa, Artificial Sequence
  Description of Artificial Sequence 9GD Norleu-AII(1-7)
DRXYIHP

SEQ ID NO: 42 - protein, 8 aa, Artificial Sequence
  Description of Artificial Sequence GSD28 Ile8-AII
DRVYIHPI

SEQ ID NO: 43 - protein, 8 aa, Artificial Sequence
  Description of Artificial Sequence Ala3aminoPhe6-AII
DRAYIXPF

SEQ ID NO: 44 - protein, 7 aa, Artificial Sequence
  Description of Artificial Sequence Ala3-AIII
RVAIHPF

SEQ ID NO: 45 - protein, 8 aa, Artificial Sequence
  Description of Artificial SequenceGly1-AII
GRVYIHPF

SEQ ID NO: 46 - protein, 8 aa, Artificial Sequence
  MOD_RES (4): Nle
RVYXLHPF

SEQ ID NO: 47 - protein, 8 aa, Artificial Sequence
  Description of Artificial Sequence Acpc3-AII
DRXYIHPF

SEQ ID NO: 48 - protein, 8 aa, Artificial Sequence
  Description of Artificial Sequence Orn2-AII
DXVYIHPF

SEQ ID NO: 49 - protein, 8 aa, Artificial Sequence
  Description of Artificial Sequence Citron2-AII
DXVYIHPF

SEQ ID NO: 50 - protein, 7 aa, Artificial Sequence
  Description of Artificial Sequence Pro3Ala4-AII(1-7)
DRPAIHP